(19) United States
(12) Patent Application Publication
(10) Pub. No.: US 2009/0221715 A1
(43) Pub. Date: Sep. 3, 2009

(54) NOVEL POLYMORPH OF ATOVAQUONE

(76) Inventors: Ashok Kumar, Mumbai (IN); Suneel Yeshwant Dike, Mumbai (IN); Pramilkumar Mathur, Mumbai (IN); Nellithanath Thankachen Byju, Mumbai (IN); Brajesh Sharma, Mumbai (IN); Swapnil Shreekant Kore, Mumbai (IN); Vitthal Suryabhan Buchude, Mumbai (IN); Dharmendra Singh, Mumbai (IN)

Correspondence Address: BLANK ROME LLP, WATERGATE, 600 NEW HAMPSHIRE AVENUE, N.W., WASHINGTON, DC, 20037, US

(21) Appl. No.: 12/394,419

(22) Filed: Feb. 27, 2009

(30) Foreign Application Priority Data
Feb. 28, 2008 (IN) 408/MUM/2008

(51) Int. Cl. A61K 31/122; C07C 49/303

(52) U.S. Cl. 514/689; 568/337

(57) ABSTRACT

The present invention relates to a novel polymorphic form of atovaquone. More particularly, it relates to a novel crystalline form, that has improved solubility and other bulk characteristics suitable for pharmaceutical application. The present invention also relates to processes for preparing a new polymorphic form of atovaquone and its use in industry.

The present invention relates to a novel polymorphic form of atovaquone. More particularly, it relates to a novel crystalline form, that has improved solubility and other bulk characteristics suitable for pharmaceutical application. The present invention also relates to processes for preparing a new polymorphic form of atovaquone and its use in industry.

### BACKGROUND OF THE INVENTION

Atovaquone, chemical name being trans-2-[4-(4-chlorophenyl)cyclohexyl]-3-hydroxy-1,4-naphthoquinone, is a hydroxy-1,4-napthoquinone, an analog of ubiquinone, with antipneumocystic activity. Atovaquone is potently active (in animals and in vitro) against Pneumocystis carinii, Plasmodia, and tachyzoite and cyst forms of Toxoplasma gondii. Due to its inhibitory effect in sensitive parasites, atovaquone can act by selectively affecting mitochondrial electron transport and parallel processes such as ATP and pyrimidine biosynthesis. Atovaquone is a useful medicine for the treatment and prophylaxis of Pneomocystis carinii infections.

Atovaquone is the trans-isomer of 2-[4-(4-chlorophenyl)cyclohexyl]-3-hydroxy-1,4-naphthoquinone whose synthesis, activity and uses are disclosed in the patent Nos. U.S. Pat. No. 5,053,432 and EP0362996.

There are few reports available for the preparation of atovaquone exploring various synthetic alternatives. However, on the production of various crystalline forms of atovaquone is disclosed in WO2006008752 (WO '752). WO '752 discloses that atovaquone can exist in three different polymorphic forms (designated as Form I, Form II and Form III) and provided analytical characterization for those polymorphs. The product obtained by the basic molecule patent was characterized for the first time in this publication, and designated as Form I. The stability data of the above forms are not reported.

There are other reports on microparticles of atovaquone. For example, U.S. Pat. Nos. 6,018,080 and 6,649,659 disclose such particles and processes for producing the same. Those microparticles of atovaquone have been ascribed to have increased bioavailability. It has been also narrated that the U.S. Pat. No. 5,053,432 process yielded macroparticles of atovaquone that are not suitable to be administered, even after conventional milling, due to poor solubility of the crystals in common organic/aqueous solvents.

Therefore there is a need in the art to for new forms of atovaquone, which have better solubility and improved bioavailability for making suitable dosage forms for pharmaceutical application.

### SUMMARY OF THE INVENTION

It has been seen that the crystalline forms disclosed in the prior art are substantially coarser crystals and the solubility of those forms are found to be very poor. It has now surprisingly been found that the atovaquone crystals can occur in a structurally different physical form. Thus the present invention provides atovaquone in a substantially pure polymorphic form (hereinafter referred to as the compound of the invention). The novel polymorph can be obtained as a well defined compound and is herein after designated as “Form IPCA-ATO”. The character of the new form can be defined either by distinct peaks in its powder X-Ray pattern, distinct endotherms in its DSC, or peaks in its IR spectrum.

The present invention also provides a process to obtain and a method of differentiating the novel form of atovaquone from other forms of atovaquone. The compound of the invention is advantageous because it is found to contain stable and well defined crystals with lower bulk density than the corresponding morphologically different atovaquone compounds of the prior art. As such, the compound of the present invention has better solubility properties leading to higher bioavailability. The compound of the invention is also easier to characterize because it exists in a well defined state. Because it is useful for pharmaceutical application, the invention also includes pharmaceutical compositions containing the compound of the present invention.

### BRIEF DESCRIPTION OF THE DRAWINGS

FIG. 1 shows an X-ray powder diffractogram of an exemplary batch of “Form IPCA-ATO” of atovaquone obtained in accordance with the invention.

FIG. 2 shows infrared (IR) spectra of “Form IPCA-ATO” of atovaquone obtained in accordance with the invention.

FIG. 3 shows a differential scanning calorimetry (DSC) analysis diagram of an exemplary batch of “Form IPCA-ATO” of atovaquone obtained in accordance with the invention

### DETAILED DESCRIPTION OF THE PREFERRED EMBODIMENTS

Unless specified otherwise, all technical and scientific terms used herein have the same meaning as commonly understood by one of ordinary skill in the art, to which this invention belongs. Although any methods and materials similar or equivalent to those described herein can be used in the practice or testing of the present invention, the preferred methods and materials are described. To describe the invention, certain terms are defined herein specifically as follows.

Unless stated to the contrary, any of the words “having”, “including,” “includes,” “comprising,” and “comprises” mean “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims.

The term “isolating” is used to indicate separation or collection or recovery of the compound of the invention being isolated in the specified form.

The term “separating from a solvent” with respect to the solids described herein means obtaining a solid of specified characteristics from a solution or a partial solution.

The term “treating” means adding or combining or mixing the stated reagent or materials to the things being treated.

The term “forming a solution” means obtaining a solution of a substance in a solvent in any manner. It encompasses partial solutions.

The term “stable” as used herein, refers to the tendency to remain substantially in the same physical form for at least a month, preferably at least 6 months, more preferably at least a year, still more preferably at least 3 years, when stored under ambient conditions (20° C./60% RH) without external treatment. Substantially the same physical form in this context means that at least 70%, preferably at least 80% and more preferably at least 90% of the crystalline form remains.

For the purposes of this description and claims of the present invention, the phrase “atovaquone ‘Form IPCA-ATO’” refers to the novel form of atovaquone, wherein the ‘IPCA-ATO’ is referring to a crystalline form of Atovaquone that one of skill in the art can identify as a distinct entity distinguishable from other crystalline forms of atovaquone based on the characterization details provided herein with the present invention.

As used herein, the phrase having “at least one characteristic of ‘Form IPCA-ATO’”, refers to a crystalline form of atovaquone that possesses at least one of the characteristic PXRD peaks or distinct peaks in IR spectrum provided herein. For example, a single or a combination of PXRD peaks which are not found in another crystalline form of Atovaquone is enough to show at least one of the characteristics of “Form IPCA-ATO” of Atovaquone, the compound of the present invention. A single or a combination of peaks in an FT IR spectrum provided herein with this invention may also serve the same purpose.

Identification of solids obtained by the present invention can be made by methods known in the art, such as X-Ray powder diffraction (XRPD), Fourier Transform Infrared (FT-IR) spectra, and differential scanning calorimetry (DSC). Of course, it should be understood that operator, instrument and other similar changes may result in some margin of error with respect to analytical characterization of the solid.

The FTIR, DSC and XRPD methods used for the identification and characterization of the novel form of atovaquone are described below:

### a) FT-IR Spectral Analysis

FTIR spectra of novel form was recorded directly on untreated powder by means of spectrometer. Spectra was recorded at room temperature from 4000 cm−1 to 650 cm−1, for each sample 32 scans were collected at a resolution of 4 cm−1.

### b) XRPD Studies

Analytical characterization of the compound according to the invention was carried out by using X-ray powder diffraction using a PANalytical XpertPRO X-Ray machine of Philips make. The X-ray powder diffraction patterns were recorded with Cu K alpha-1 radiation source (voltage of 45 kV; current: 40 mA). The step scan mode was performed with a step size of 0.008°, at a scan rate of 14.59 step/s.

### c) DSC

DSC analysis of the novel form was recorded at a heating rate of 10° C. per minute at a temperature range from 50° C. to 250° C.

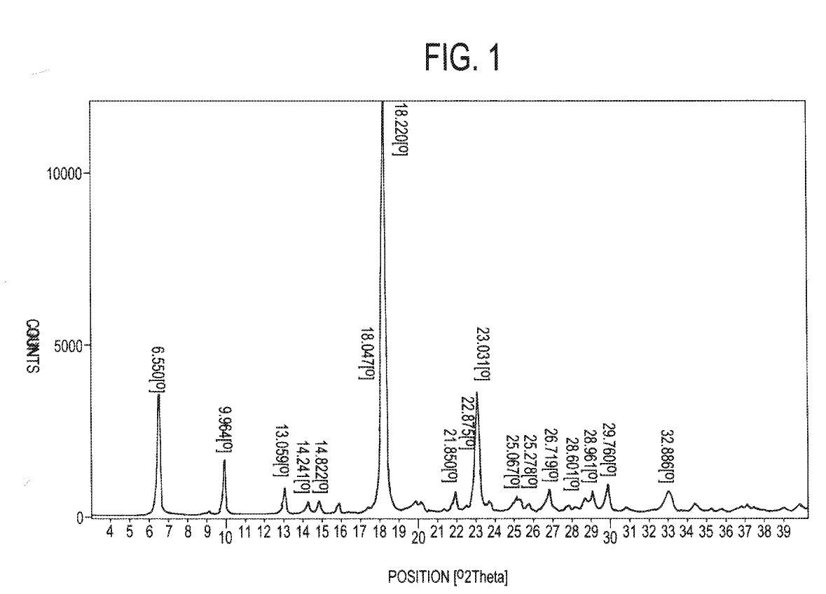

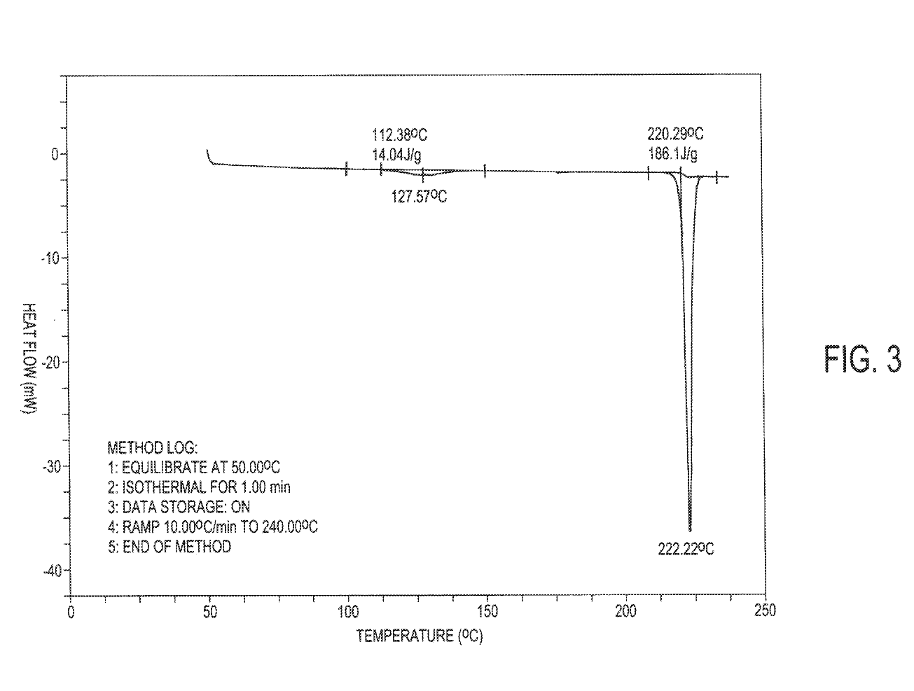

The compound of the invention is characterized by the positions of the major peaks in the X-ray powder diffractogram, but may also be characterized by conventional FT-IR spectroscopy and endotherms in DSC diagram. These characteristics are not exhibited by any other form of atovaquone and accordingly, the “Form IPCA-ATO” of the present invention is easily distinguishable from any other crystal form of the atovaquone disclosed in prior art. Thus, the character of this new form (“Form IPCA-ATO”) is confirmed either by PXRD patterns, DSC endotherms and FT IR spectra obtained from a sample thereof which are provided as FIGS. 1 to 3 respectively. The PXRD pattern shows at least one characteristic and exclusive peak at about 6.66±0.2 and about 10.05±0.2 degrees 2θ angles. More particularly the PXRD pattern shows characteristic and exclusive peaks at 6.66±0.2, 10.05±0.2, 13.11±0.2, 18.27±0.2, and 23.10±0.2 degrees 2θ angles.

The novel form of atovaquone “Form IPCA-ATO” is also characterized by FT-IR spectra having peaks at about 3369, about 2935, about 1633, about 1383, about 1338, about 1312, about 1231 and about 1053 cm⁻¹, which are characteristic for the present form.

The novel form of atovaquone “Form IPCA-ATO” is also characterized by endotherms in a DSC. Thermal analysis results in a differential scanning calorimeter thermogram taken at a heating rate of 10° C. per minute in a open pan that exhibits a melting endotherm with a peak temperature of about 117-130° C. (an onset temperature in the range of about 100-120° C.), and a second endotherm having peak at about 220-222° C. (onset temperature in the range of about 217-219° C.). The position of the first endotherm can shift the position depending upon the heating rate.

The XRPD main peaks, with positions and relative intensities, have been extracted from the diffractogram in FIG. 1 and are given below in table 1. The relative intensities are less reliable, as it can vary considerably and some additional very weak peaks found in the diffractogram have been omitted from table 1.

TABLE 1

| 2θ values in | | Percentage relative |
|---|---|---|
| degrees | d spacing | intensity |
| 6.6 | 13.42 | 30.06 |
| 9.9 | 8.8 | 12.58 |
| 13.1 | 6.7 | 6.36 |
| 18.2 | 4.8 | 100.00 |
| 23.0 | 3.8 | 28.93 |
| 32.9 | 2.7 | 4.87 |

In a further aspect, the present invention provides processes for the preparation of the atovaquone “Form IPCA-ATO” which comprises; i) contacting atovaquone of any physical form in an organic solvent to obtain a solution at a suitable temperature for a suitable time; ii) subjecting it to rapid chilling; and iii) recovering the novel form from the reaction solution. “Rapid chilling,” as mentioned herein, refers to cooling a rate in the range of about 5° C. to 50° C. per minute, and preferably about 20-50° C. per minute.

In one embodiment of the present invention, preparation of “Form IPCA-ATO” comprises i) subjecting a solution of atovaquone to chilling either in a cold bath of liquid nitrogen or dry ice prepared in a suitable solvent medium until frozen and removing the solvent from the mass thus obtained to recover the novel form of atovaquone. Alternately the atovaquone solution may be added to either liquid nitrogen or dry ice to precipitate the new form followed by removal of the solvent. The solvent may be removed by distillation or under vacuum in a lyophilizer. The organic solvents may be selected from, but not limited to, chlorinated solvent, especially dichloromethane.

According to a second embodiment, “Form IPCA-ATO” can be prepared by a process comprises i) cooling a solution of atovaquone in a solvent like chloroform to a temperature below 5° C., more preferably below 0° C.; and ii) adding an anti-solvent, such as methanol, and removing the solvent and anti-solvent from the mass thus obtained to recover the novel form of atovaquone. Alternately, the atovaquone solution may be prepared in a mixture of dichloromethane and dimethyl formamide and chilled to a lower temperature, preferably below −10° C., to crystallize the novel form, followed by removal the solvent. The solvent and/or anti-solvent may be removed by distillation or in a lyophilizer.

“Suitable temperature” as used herein is a temperature which the solution can be formed and be able to induce the transformation of atovaquone into the novel form. Examples of such suitable temperatures include, but are not limited to, room temperature, preferably lower than room temperature, still preferably less than about 0° C. and more preferably less than about −30° C.

“Suitable time” as used herein is a time that results in better conversion of the starting material into novel crystalline form without causing any decomposition of either compounds, i.e. results in a good yield. This suitable time will vary depending on the mode of chilling used, can be established by routine experimentation. The faster the rate of cooling, the shorter time is needed to give the desired conversion. The amount of solvent is not crucial and will depend on the process conversion and conditions desired. To have complete conversion to the novel form of the present invention, complete dissolution of atovaquone in the selected solvent is desired. Process conditions for certain embodiments are further illustrated in the Examples.

Atovaquone has been indicated for use in the following indications: Pneumocystis carinii, Plasmodia, and tachyzoite and cyst forms of Toxoplasma gondii. It may be used alone or concomitantly with other classes of agents like mefloquine or proguanil (anti-malarials).

In a further aspect, the invention thus provides atovaquone “Form IPCA-ATO” for use in treating Pneumocystis carinii, Plasmodia, and tachyzoite and cyst forms of Toxoplasma gondii, either alone or in combination with other anti-malarial agents. In the practice of the invention, the most suitable route of administration as well as the magnitude of a therapeutic dose of atovaquone “Form IPCA-ATO” in any given case will depend on the nature and severity of the disease to be treated. The dose, dose frequency may also vary according to the age, body weight and response of the individual patient.

The invention also provides pharmaceutical compositions containing atovaquone “Form IPCA-ATO” which may optionally contain other crystalline forms and/or other active pharmaceutical drugs. In addition to the active ingredient(s), the pharmaceutical compositions of the present invention can contain one or more commonly used pharmaceutical excipients. Excipients are added to the composition for a variety of purposes as known to one skilled in the art.

The bulk density of the new form was compared with other crystalline forms and found that the atovaquone “Form IPCA-ATO” is lighter than the other forms. The results are summarized in Table 2.

TABLE 2

| | | Bulk | Tapped |
|---|---|---|---|
| Ser. No. | Sample name | density g/ml | density g/ml |
| 1 | Form I (ATO-8094P) | 0.4802 | 0.6402 |
| 2 | Form III (ATO-8110) | 0.2975 | 0.4010 |
| 3 | Form IPCA-ATO (ATO-8099) | 0.2353 | 0.3801 |

The starting atovaquone may be obtained by following any known process disclosed in the literature.

Without further description, it is believed that one of ordinary skill in the art can, using the preceding description and the following illustrative examples, make and utilize the compounds of the present invention and practice the claimed methods. The following examples are given to illustrate the present invention. It should be understood that the invention is not to be limited to the specific conditions or details described in those examples.

### EXAMPLE 1

1.0 grams of atovaquone (Form I) was taken in 35 ml of dichloromethane at room temperature. It was dissolved completely and filtered out any undissolved particles. The solution was then chilled on a nitrogen bath until the dichloromethane solution solidified. The material was lyophilized and dichloromethane was removed completely to obtain the novel crystalline form. Yield 1.0 gm. The XRPD, IR spectra, and DSC of the sample were recorded and are reproduced in FIGS. 1 to 3.

### EXAMPLE 2

1.0 grams of atovaquone (Form I) was taken in 35 ml of dichloromethane at room temperature. It was dissolved completely and filtered out of any undissolved particles. The solution was poured on liquid nitrogen in another vessel until the dichloromethane solution solidified. The solid obtained lyophilized and dichloromethane was removed completely to obtain the novel crystalline form. Yield 1.0 gm. The XRPD, IR spectra, and DSC of the sample were recorded and are similar to those of FIGS. 1 to 3.

### EXAMPLE 3

0.5 grams of atovaquone (Form I) was dissolved in 15 ml of chloroform at room temperature and cooled to 0° C. 20 ml methanol was added drop-wise at 0° C.; and the precipitate obtained was filtered to obtain the new crystalline form. Yield 80%. The XRPD, IR spectra, and DSC of the sample were recorded and are similar to those of FIGS. 1 to 3.

### EXAMPLE 4

0.5 grams of atovaquone (Form I) was dissolved in 7.5 ml of dichloromethane at room temperature and 2.5 ml dimethyl formamide was added. The solution was filtered and added to a reaction flask maintained at −20° C., stirred for 1 hour and filtered to obtain the new crystalline form. Yield 80%. The XRPD, IR spectra, and DSC of the sample were recorded and are similar to those of FIGS. 1 to 3.

### EXAMPLE 5

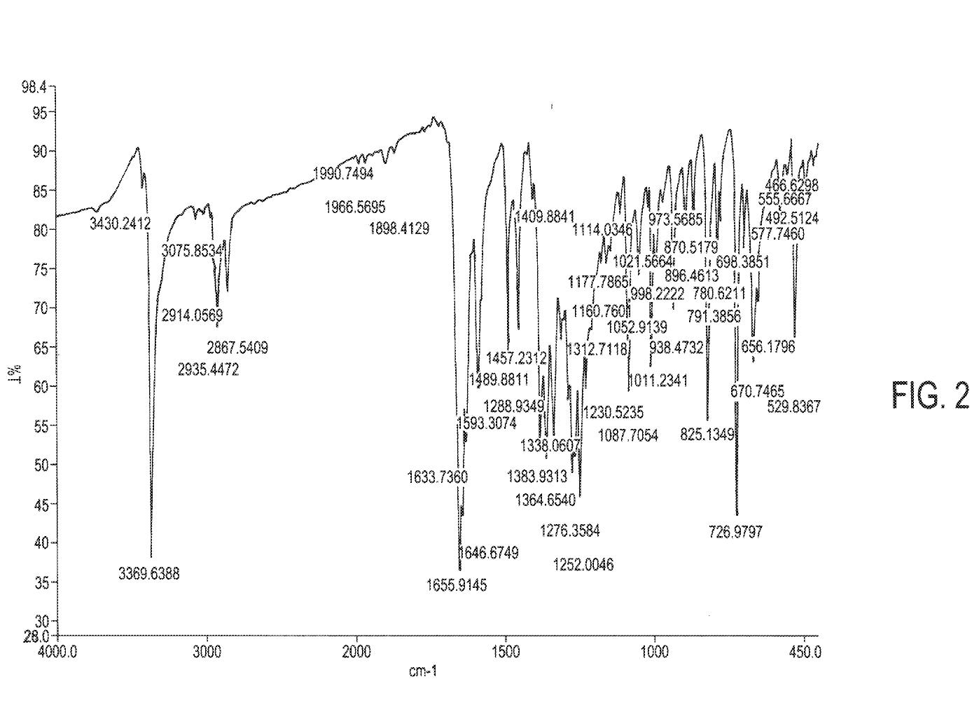

35 grams of atovaquone (Form I) was dissolved in 1575 ml of dichloromethane at room temperature, filtered and added to a reaction flask pre-chilled to −20° C. The solvent was partly distilled under vacuum while maintaining the temperature at −20° C., and filtered to obtain the new crystalline form. Yield 86.8%. The XRPD and IR spectra of the sample were recorded and are similar to those of FIGS. 1 to 2. DSC shows endotherm at 128.83 and 222.27° C.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A crystalline form of atovaquone having X-Ray powder diffraction (XRPD) pattern containing a peak at about 10.05 degrees 2θ angles.

2. The crystalline form of atovaquone of claim 1, wherein the powder XRPD pattern further containing peaks at about 6.66, about 13.11, about 18.27, and about 23.10 degrees 2θ angles.

3. The crystalline form of atovaquone of claim 1, further having FT-IR spectra containing peaks at about 3369, about 2935, about 1633, about 1383, about 1338, about 1312, about 1231, and about 1053 cm⁻¹.

4. The crystalline form of atovaquone of claim 1, further having a differential scanning calorimetry (DSC) containing a first endotherm with an onset temperature in the range of about 100-120° C. and second endotherm with an onset temperature in the range of about 217-219° C.

5. The crystalline form of atovaquone of claim 1, having a bulk density of about 0.2353 g/mL.

6. The crystalline form of atovaquone of claim 1, having a tapped density of about 0.0.3801 g/mL.

7. A crystalline form of atovaquone having FT-IR spectra containing peaks at 3369, 2935, 1633, 1383, 1338, 1312, 1231, and 1053 cm⁻¹.

8. A crystalline form of atovaquone having differential scanning calorimetry (DSC) containing a first endotherm with an onset temperature in the range of about 100-120° C. and second endotherm with an onset temperature in the range of about 217-219° C.

9. A pharmaceutical composition comprising the novel crystalline form of atovaquone of claim 1.

10. The pharmaceutical composition of claim 9, further comprising proguanil or its pharmaceutical salt.

11. A method for preparing crystalline form of atovaquone of claim 1, comprising the steps of
i) dissolving atovaquone of any physical form in an organic solvent to obtain a solution;
ii) cooling said solution; and
iii) recovering the crystalline form of atovaquone of claim 1 from the reaction solution.

12. The method of claim 11, wherein the recovering step comprises distillation or lyophilizing.

13. The method of claim 11, wherein the solvent is dichloromethane, chloroform, dimethyl formamide, or mixtures thereof.

14. The method of claim 11, further comprising the step of adding an antisolvent to the cooled solution to crystallize the crystalline form of atovaquone of claim 1.

15. The method of claim 14, wherein the antisolvent is methanol.

16. The method of claim 14, wherein the antisolvent in added at a temperature of less than about 0° C.

17. The method of claim 11, wherein step (b) occurs at less than about 0° C.

18. The method of claim 11, wherein step (b) occurs at less than about −30° C.

19. The method of claim 11, wherein step (b) comprises rapidly cooling the solution to crystallize the crystalline form of atovaquone of claim 1.

20. The method of claim 11, wherein step b) comprises chilling the solution either in a cold bath of liquid nitrogen or a dry ice bath until frozen.
